(19) United States
(12) Patent Application Publication
(10) Pub. No.: US 2010/0056485 A1
(43) Pub. Date: Mar. 4, 2010

(54) NANOSOAP CONTAINING SILVER NANOPARTICLES

(75) Inventor: Seung Bum Park, Seoul (KR)

Correspondence Address: Seung Bum Park, 1221-1 502 Seoul National University Faculty Apt., Bongchun-dong, Gwanak-gu, Seoul, KR

(73) Assignee: SNU R&DB Foundation

(21) Appl. No.: 12/200,817

(22) Filed: Aug. 28, 2008

(51) Int. Cl. A61K 31/661; A61K 31/555; A61P 31/04; A61P 31/12; A61P 31/10; A61K 31/28

(52) U.S. Cl. 514/120; 514/184; 514/495

(57) ABSTRACT

Disclosed herein is a silver nanoparticle based antimicrobial composition and methods for making the same. The antimicrobial composition comprises an amphiphilic molecule having at least one hydrophilic group and at least hydrophobic groups attached thereto; and an at least one silver nanoparticle in contact with the amphiphilic molecule. Also disclosed are uses for the antimicrobial composition such as for the treatment of skin disorders.

### FIELD

Disclosed herein is an antimicrobial composition comprising at least one silver nanoparticle and methods for making the same. Also disclosed are uses for the antimicrobial composition such as for the treatment of skin disorders.

### BACKGROUND

Silver nanoparticles are highly toxic for microorganisms, but have relatively low toxicity for human tissue cells. Silver is also extremely active in small quantities. For certain bacteria, as little as one part per billion of silver may be effective as an antibacterial in preventing cell growth. Silver nanoparticles have shown excellent efficacy as an antimicrobial when used to treat wounds and burns. Using silver nanoparticles as an antimicrobial is particularly attractive because the microbes are not able to mutate to avoid its antimicrobial effect and thus are unable to build resistance.

Very few methods have been developed for the delivery of such particles to an infected area in order to treat bacterial and microbial infections. In addition, none of the known delivery methods are able to address the main source of resistant bacterial infections-contamination from hospitals and other healthcare facilities.

### SUMMARY

Disclosed herein is an antimicrobial composition comprising at least one silver nanoparticle and methods for making the same. Also disclosed are uses for the antimicrobial composition such as for the treatment of skin disorders.

Disclosed herein is an antimicrobial composition, comprising:
at least one amphiphilic molecule comprising at least one hydrophilic group and at least one hydrophobic group attached thereto via a linker; and
at least one silver nanoparticle in contact with the amphiphilic molecule.

In one embodiment, the antimicrobial composition further comprises a carrier.

In some embodiments, the amphiphilic molecule has at least two hydrophobic groups.

In some embodiments, the silver nanoparticle is bonded to the amphiphilic molecule. In certain embodiments, the silver nanoparticle is bonded to the amphiphilic molecule through a heterocyclic or heteroaryl linker. In some embodiments, the silver nanoparticle is bonded to the at least one hydrophobic group through a heterocyclic or heteroaryl linker.

In some embodiments, the antimicrobial composition disclosed herein is formulated for topical administration to skin of a mammalian subject. In one embodiment, the antimicrobial composition is included in a topical cosmetic formulation. In another embodiments, the antimicrobial composition is included in a topical pharmaceutical formulation.

Also disclosed herein is a molecule, comprising:
at least one amphiphilic molecule comprising at least one hydrophilic group and at least one hydrophobic group attached thereto via a linker; and
at least one silver nanoparticle in contact with the amphiphilic molecule, wherein the molecule is an antimicrobial.

Also disclosed herein is a method for making an antimicrobial composition, comprising contacting an amphiphilic molecule comprising at least one hydrophilic group and at least one hydrophobic group attached thereto via a linker having at least one first reactive functional group attached to the at least one hydrophilic group or the at least one hydrophobic group; with at least one silver nanoparticle having a second reactive functional group attached thereto under reaction conditions suitable to form the antimicrobial composition.

The antimicrobial compositions disclosed herein can be used to prevent or to treat a bacterial infection in a mammalian subject by administering to the subject an effective amount of the antimicrobial composition. Other medical conditions can be prevented or treated including, but not limited to, acne; a burn; a sunburn; a chemical burn; a rash; a lesion; a scrape; a blister; scale; an abscess; a sore; and disorders resulting therefrom.

Also disclosed herein is a kit comprising:
a) an antimicrobial composition as disclosed herein; and
b) instructions for the use of the antimicrobial composition.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1, panels A to D, show a schematic of illustrative embodiments of the disclosed antimicrobial composition wherein two hydrophobic groups are attached to the hydrophilic group.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

### Abbreviations and Definitions

Unless otherwise stated all temperatures are in degrees Celsius (° C.). Also, in these examples and elsewhere, abbreviations have the following meanings:

TABLE 1
Abbreviations

| Abbreviation | Term |
|---|---|
| PEG | polyethylene glycol |
| mg | milligram |
| kg | kilogram |
| TBTA | tris[(1-benzyl-1H-1,2,3-triazol-4-yl)methyl]amine |

As used herein, certain terms may have the following defined meanings.

As used herein, the term “comprising” means that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps for preparing the microfluidic device. Embodiments defined by each of these transition terms are within the scope of the present technology.

As used herein, the term “antimicrobial” refers to substances or mixtures of substances capable of destroying or inhibiting the growth of microorganisms such as bacteria, viruses, and fungi. The antimicrobial compositions disclosed herein can be used on inanimate objects and surfaces as well as to treat mammals. In some embodiments, the antimicrobial composition as disclosed herein destroys or inhibits the growth of microorganisms by at least about 5%, or alternatively, at least about 10%, or alternatively, at least about 20%, or alternatively, at least about 30%, or alternatively, at least about 40%, or alternatively, at least about 50%, or alternatively, at least about 60%, or alternatively, at least about 70%, or alternatively, at least about 80%, or alternatively, at least about 90%. In one embodiment, the antimicrobial composition destroys or inhibits the growth of microorganisms by greater than about 90%.

As used herein, the term “amphiphilic” refers to an organic compound composed of hydrophilic and hydrophobic portions. Examples of such molecules are surfactants, detergents, bile salts, and phospholipids. The term “hydrophilic” refers to groups which have a strong affinity for water, and the term “hydrophobic” refers to groups which have little or no affinity for water.

As used herein, the term “attached” refers to a chemical bond wherein two molecules or groups are bonded together to form a single molecule. As used herein, the term “bonded” refers to a chemical bond. Various types of chemical bonds can be employed in the methods disclosed herein, either alone or in combination. Examples of bonds include, but are not limited to, covalent bonds, polar covalent bonds, ionic bonds and hydrogen bonds.

As used herein, the term “nanoparticle” refers to a nano scale particle. A “silver nanoparticle” is a nanoparticle comprising silver(0). In some embodiments, the silver nanoparticles disclosed herein are from about 5 nm to about 500 nm in diameter. In one embodiment, the nanoparticles are from about 100 to 500 nm in diameter.

As used herein, the term “topical administration” refers to the application of a pharmaceutical agent to the external surface of the skin. Topical administration includes application of the composition to intact skin, to broken, raw or open wound of skin. Topical administration of a pharmaceutical agent can result in a limited distribution of the agent to the skin and surrounding tissues or, when the agent is removed from the treatment area by the bloodstream, can result in systemic distribution of the agent.

As used herein, the term “alkyl” refers to a saturated monovalent hydrocarbyl group having from 1 to 50 carbon atoms, more particularly from 6 to 25 carbon atoms. The alkyl group can be branched or linear. This term is exemplified by groups such as methyl, ethyl, n-propyl, iso-propyl, n-butyl, t-butyl, n-pentyl, n-hexyl, n-heptyl, n-octyl, n-nonyl, n-decyl, n-dodecyl, and the like.

As used herein, the term “cycloalkyl” refers to cyclic alkyl groups of from 3 to 10 carbon atoms having single or multiple cyclic rings including, by way of example, adamantyl, cyclopropyl, cyclobutyl, cyclopentyl, cyclooctyl and the like.

As used herein, the term “alkenyl” refers to a hydrocarbyl group preferably having from 2 to 8 carbon atoms and having from 1 to 2 sites of alkenyl unsaturation.

As used herein, the term “alkynyl” refers a hydrocarbyl group preferably having from 2 to 8 carbon atoms and having from 1 to 3 sites of alkynyl unsaturation.

As used herein, the term “aryl” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is the aryl group.

As used herein, the term “heteroaryl” refers to an aromatic ring of from 1 to 15 carbon atoms, preferably from 1 to 10 carbon atoms, and 1 to 4 heteroatoms within the ring selected from the group consisting of oxygen, nitrogen, and sulfur. Such heteroaryl groups can have a single ring (e.g., pyridinyl, furyl, triazole or thienyl) or multiple condensed rings (e.g., indolizinyl or benzothienyl) provided the point of attachment is through a ring containing the heteroatom and that ring is aromatic. The nitrogen and/or sulfur ring atoms can optionally be oxidized to provide for the N-oxide or the sulfoxide, and sulfone derivatives. Examples of heteroaryls include but are not limited to, pyridinyl, pyrrolyl, pyrazolyl, indolyl, thiophenyl, thienyl, furanyl, oxazolyl, triazolyl, benzoxazolyl, benzimidazolyl, benzotriazolyl; benzothiadiazolyl; benzofuroxanyl, quinolinyl, quinolidinyl, quinazolinyl, benzotriazinonyl, tetrazolyl and fused heteroaryls of above mentioned heteroaryls.

As used herein, the term “heterocyclic” refers to a saturated or unsaturated nonaromatic group having a single ring or multiple condensed rings, from 1 to 10 carbon atoms and from 1 to 4 hetero atoms selected from the group consisting of nitrogen, sulfur or oxygen within the ring wherein, in fused ring systems, one or more the rings can be aryl or heteroaryl. Non-limiting examples include isoxazolidinyl, isoxazolidinonyl, pyranyl, benzodiazepinyl, benzopyranyl, benzazepinyl; and fused heterocycles of above mentioned heterocycles.

As used herein, the term “alkoxy” refers to the group “alkyl-O—” which includes, by way of example, methoxy, ethoxy, n-propoxy, iso-propoxy, n-butoxy, t-butoxy, sec-butoxy, n-pentoxy, and the like.

As used herein, the term “thioalkoxy” refers to the group “alkyl-S—”.

As used herein, the term “carboxy ester” refers to the groups —C(═O)—O-alkyl, —C(═O)—O-cycloalkyl, —C(═O)—O-aryl, —C(═O)—O-heteroaryl, —C(═O)—O-heterocyclic.

As used herein, the term “thiocarboxy ester” refers to the groups —C(═O)—S-alkyl, —C(═O)—S-cycloalkyl, —C(═O)—S-aryl, —C(═O)—S-heteroaryl, —C(═O)—S-heterocyclic.

As used herein, the terms “halo” and “halogen” refer to fluorine, chlorine, bromine and iodine.

As used herein, the term “oxo” refers to the group (═O) or (—O—).

As used herein, the term “thioxo” refers to the group (═S) or (—S—).

As used herein, the term “hydroxy” refers to the group —OH.

As used herein, the term “nitro” refers to the group —NO₂.

As used herein, the term “amino” refers to the group —NH₂.

As used herein, the term “cyano” refers to the group —CN.

As used herein, the term “azide” refers to the group —N₃.

As used herein, the term “silyl” refers to the group —SiR₃, wherein each R is independently hydrogen, (C₁-C₆)alkyl or (C₆-C₁₂)aryl.

As used herein, the term “polyethyleneglycol (PEG)” refers to a polyether of the formula —(OCH₂CH₂)ₙOH, wherein n can vary greatly depending on the composition. For example, the PEG can have a molecular weight of about 100 to about 1000 g/mol.

As used herein, the term “carboxylate” refers to the group —CO₂—.

As used herein, the term “carboxyl” refers to the group —CO₂H.

As used herein, the term “sulfonate” refers to the group —S(═O)₂O⁻.

As used herein, the term “sulfonyl” refers to the group —S(═O)₂OH.

As used herein, the term “sulfate” refers to the group —O—S(═O)₂O⁻.

As used herein, the term “sulfinate” refers to the group —S(═O)O—.

As used herein, the term “sulfinyl” refers to the group —S(═O)OH.

As used herein, the term “phosphate” refers to the group —OP(═O)(OH)O—.

As used herein, the term “phosphinate” refers to the group —P(═O)(R)O—, wherein R is a (C₁-C₆)alkyl or (C₆-C₁₂)aryl.

As used herein, the term “phosphinyl” refers to the group —P(═O)(R)OH, wherein R is a (C₁-C₆)alkyl or (C₆-C₁₂)aryl.

As used herein, the term “phosphonate” refers to the group —P(═O)(OH)O— or —O—P(═O)RO —, wherein R is a (C₁-C₆)alkyl or (C₆-C₁₂)aryl.

As used herein, the term “phosphonyl” refers to the group —P(═O)(OH)₂ or —O—P(═O)RO⁻, wherein R is a (C₁-C₆)alkyl or (C₆-C₁₂)aryl.

As used herein, the term “secondary amino” refers to the group —NHR, wherein R is a (C₁-C₆)alkyl or (C₆-C₁₂)aryl.

As used herein, the term “tertiary amino” refers to the group —NR₂, wherein R is a (C₁-C₆)alkyl or (C₆-C₁₂)aryl.

As used herein, the term “quaternary amino” refers to the group —NR₃⁺, wherein each R is independently hydrogen, (C₁-C₆)alkyl or (C₆-C₁₂)aryl. Also included are quaternary amines derived from aromatic amines, such as pyridinium.

As used herein, the term “functional group” refers to atoms or small groups of atoms (e.g., two to five) that exhibit a characteristic reactivity when treated with certain reagents. This term is exemplified by groups such as alkyl optionally substituted with from 1-3 R⁴ groups, alkenyl optionally substituted with from 1-3 R⁴ groups, alkynyl optionally substituted with 1 R⁴ group, amino, azide, cyano, halo, nitro, silyl, aryl optionally substituted with from 1-4 R⁴ groups, heteroaryl optionally substituted with from 1-3 R⁴ groups, alkoxy, thioalkoxy, carboxy ester, thiocarboxy ester and polyethyleneglycol (PEG), wherein

each R⁴ is independently selected from the group consisting of oxo, thioxo, hydroxyl, amino, azide, cyano, halo, nitro, silyl, alkoxy, thioalkoxy, carboxy ester and thiocarboxy ester. In some embodiments, the functional group is alkynyl, cyano or azide.

As used herein, the term “linker” refers to a chain comprising from 1-20 atoms and may comprise atoms or groups, such as —C—, —NR—, —O—, —S—, —S(O)—, —S(O)₂—, —C(O)—, —C(S)—, —C(NR)—, —C(O)O—, —OC(O)—, —C(O)NR—, —NRC(O)—, and the like, and combinations thereof, and wherein R is hydrogen, (C₁-C₆)alkyl or (C₆-C₁₂)aryl. The linker may also be branched to link a hydrophilic group to more than one hydrophobic group. That is, for example, the linker may comprise the groups: —CH₂—, —CH₂—CH₂—, —CH₂—O—CH₂—, —CH₂—CH₂—O—CH₂—CH₂—, —CH₂—NH—CH₂—, —CH₂—CH₂—NH—CH₂—CH₂—, —CH₂—NHC(O)—CH₂—, —CH₂—C(O)NH—CH₂—, —CH₂—C(O)—CH₂—, —CH₂—OC(O)—, —CH₂—CH₂—OC(O)—, —CH₂—CH(—OC(O)—)(—CH₂—OC(O)—), —CH₂—C(—OC(O)—)(—CH₂—OC(O)—)₂, and the like.

As used herein, the term “heterocyclic or heteroaryl linker” refers to a divalent heterocycle or heteroaryl as defined herein. The points of attachment can be on a carbon atom or heteroatom of the heterocycle or heteroaryl moiety.

The term “reaction conditions” refers to conditions which comprise solvent (if required), time, temperature, pressure, concentration, and the like. It is well known to those skilled in the art that the reaction conditions may vary depending on the components which are being reacted.

As used herein, the term “non-toxic” refers to a compound that does not cause adverse health effects or harm to a mammal, whether exposed orally, intravenously or dermally, in the required active concentration. In some embodiments, a compound is considered non-toxic based on the LD₅₀ (median lethal dose by oral or dermal exposure) or LC₅₀ (median lethal inhalation concentration for a one-hour exposure) value. It is suggested that non-toxic reagents have an oral LD₅₀ of greater than about 500 mg/kg or a dermal LD₅₀ of greater than about 1000 mg/kg.

### The Antimicrobial Composition Comprising a Silver Nanoparticle

Disclosed herein is an antimicrobial composition, comprising:
at least one amphiphilic molecule comprising at least one hydrophilic group and at least one hydrophobic group attached thereto via a linker; and
at least one silver nanoparticle in contact with the amphiphilic molecule.

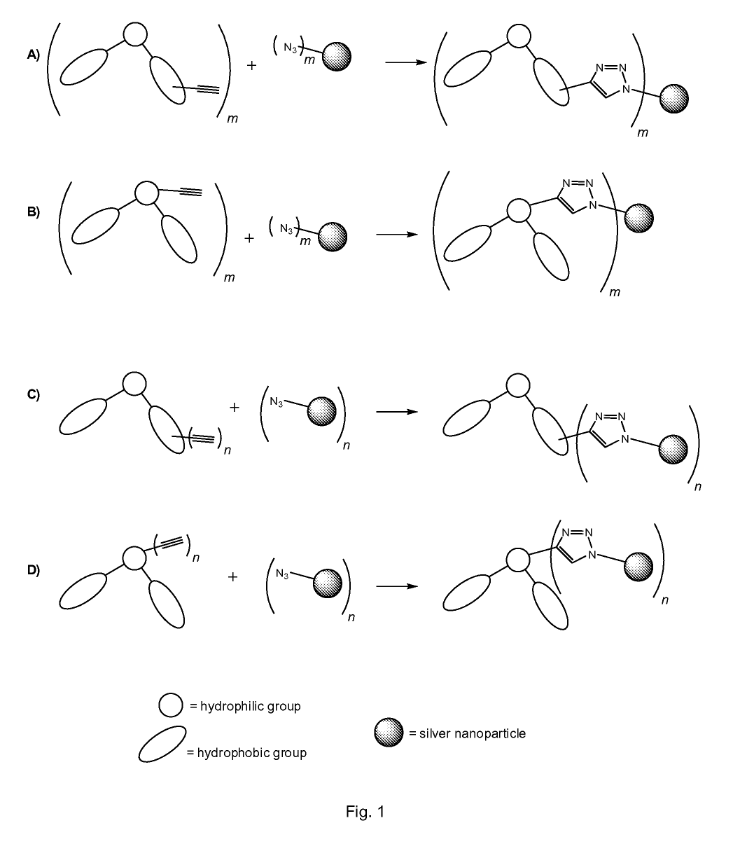

In some embodiments, as depicted in FIG. 1, the antimicrobial composition comprises at least two hydrophobic groups. In some embodiments, the molecular weight of the antimicrobial composition is from at least about 150 grams per mole to about 50,000 grams per mole. Alternatively, in other embodiments, the molecular weight is from about 500 grams per mole to about 50,000 grams per mole, or alternatively, from about 800 grams per mole to about 50,000 grams per mole, or alternatively, from about 1,000 grams per mole to about 50,000 grams per mole, or alternatively, from about 150 grams per mole to about 5,000 grams per mole, or alternatively, from about 500 grams per mole to about 4,000 grams per mole, or alternatively, from about 600 grams per mole to about 3,000 grams per mole, or alternatively, from about 800 grams per mole to about 2,000 grams per mole, or alternatively, from about 1,000 grams per mole to about 2,000 grams per mole.

The hydrophobic groups can have any configuration, such as linear, branched, cyclic or substituted, provided that they allow the formation of micelles. In general, at least six carbon atoms are required for micelle formation, which micelles are commonly found in soaps. In some embodiments, each hydrophobic group has from 8 to about 50 carbon atoms. In some embodiments, the antimicrobial composition has at least one hydrophobic group that is, independently, optionally substituted alkyl, optionally substituted alkenyl, optionally substituted alkynyl, optionally substituted cycloalkyl or optionally substituted aryl. In one embodiment, the antimicrobial composition has at least one hydrophobic group that is, independently, an optionally substituted cycloalkyl group. The substitution can be any chemical moiety so long as micelle formation is not substantially disrupted. In some embodiments, each hydrophobic group is optionally substituted with from at least about 1 to about 6 substituents selected from the group consisting of alkyl optionally substituted with from 1-3 R⁴ groups, alkenyl optionally substituted with from 1-3 R⁴ groups, alkynyl optionally substituted with 1 R⁴ group, amino, azide, cyano, halo, nitro, silyl, aryl optionally substituted with from 1-4 R⁴ groups, heteroaryl optionally substituted with from 1-3 R⁴ groups, alkoxy, thioalkoxy, carboxy ester, thiocarboxy ester and polyethyleneglycol (PEG). Each R⁴ is independently selected from the group consisting of oxo, thioxo, hydroxyl, amino, azide, cyano, halo, nitro, silyl, alkoxy, thioalkoxy, carboxy ester and thiocarboxy ester.

The hydrophilic group functions to solubilize the antimicrobial composition. In some embodiments, the hydrophilic group is selected from the group consisting of carboxyl, carboxylate; sulfonyl, sulfonate, sulfate; sulfinyl, sulfinate, phosphenyl, phosphate; phosphinyl, phosphinate; phosphonyl, phosphonate; amino, secondary amino, tertiary amino, and quaternary amino.

As depicted in FIG. 1, the silver nanoparticle can be in bonded to either the hydrophilic group or a hydrophobic group. In some embodiments, more than one amphiphilic molecule is in contact with a silver nanoparticle. Depending on the size of the amphiphilic molecule, it is contemplated that anywhere from 1 to about 10,000 amphiphilic molecules can be in contact with a silver nanoparticle. As shown in FIG. 1, panels A and B, m can be from 1 to about 10,000. In addition, more than one silver nanoparticles can be in contact with the amphiphilic molecule. In some embodiments, two silver nanoparticles in contact with the amphiphilic molecule. Alternatively, three two silver nanoparticles, or alternatively, four silver nanoparticles, or alternatively, five silver nanoparticles, or alternatively up to ten silver nanoparticles. In some embodiments, the silver nanoparticle is bonded to the at least one hydrophobic group. As shown in FIG. 1, panels C and D, n can be from 1 to about 10.

The number of silver nanoparticles can be discreet when the amphiphilic molecule has defined size and/or the mixture has an ordered secondary/tertiary structure through supramolecular interactions, such as hydrogen bonding or hydrophobic interactions. However, if the structure of the amphiphilic molecules in a mixture are not uniform or the molecules are disordered, the ratio of silver nanoparticles to amphiphilic molecules might not be discreet and would thus be an average number dispersed throughout the mixture. In some embodiments, the ratio of amphiphilic molecules to silver nanoparticles is an average of 0.1 to about 10,000.

The silver nanoparticle in the composition is provided in an amount to provide the desired anti-microbial properties. The desired antimicrobial properties in any composition is determined by its intended use. For example, if the composition described herein is for use as a dermal antimicrobial composition, it is contemplated that the amount of silver nanoparticles is the amount required to reduce a microbial infection by at least about 5%, or alternatively, at least about 10%, or alternatively, at least about 15%, or alternatively, at least about 20%, or alternatively, at least about 25%, or alternatively, at least about 30%, or alternatively, at least about 35%, or alternatively, at least about 40%, or alternatively, at least about 50%, or alternatively, at least about 60%, or alternatively, at least about 70%, or alternatively, at least about 80%, or alternatively, at least about 90%.

In some embodiments, the silver nanoparticle is bonded to the amphiphilic molecule. In some embodiments, the silver nanoparticle is bonded to the amphiphilic molecule through a heterocyclic or heteroaryl linker. FIG. 1 shows the silver nanoparticle bonded to the amphiphilic molecule through a triazole. In some embodiments, the heterocyclic or heteroaryl linker is selected from the group consisting of: triazole; tetrazole; pyrazole; isoxazolidine; pyrrole; isoxazolidinone; oxazole; pyran; furan; benzodiazepine; benzopyran; benzoxazole; indole; benzimidazole; benzotriazole; benzothiadiazole; benzofuroxane; quinoline; quinolidine; quinazoline; benzotriazinone; benzazepine; and fused heterocycles of above mentioned heterocycles. In some embodiments, the silver nanoparticle is bonded to the at least one hydrophobic group through a heterocyclic or heteroaryl linker.

Also disclosed herein is an antimicrobial molecule, comprising
at least one amphiphilic molecule comprising at least one hydrophilic group and at least one hydrophobic group attached thereto via a linker; and
at least one silver nanoparticle in contact with the amphiphilic molecule.

It is contemplated that each of the embodiments disclosed herein directed to an antimicrobial composition are also applicable to the antimicrobial molecule.

### Methods of Making the Antimicrobial Composition Comprising a Silver Nanoparticle

Disclosed herein is a method for making an antimicrobial composition. The method comprises contacting an amphiphilic molecule comprising at least one hydrophilic group and at least one hydrophobic group attached via a linker having at least one first reactive functional group attached to the at least one hydrophilic group or the at least one hydrophobic group; with at least one silver nanoparticle having a second reactive functional group attached thereto under reaction conditions suitable to form the antimicrobial composition.

In some embodiments, the antimicrobial composition has at least two hydrophobic groups. In addition, in some embodiments, the antimicrobial composition has at least two silver nanoparticles. The amount of silver can be readily determined by one of skill in the art and is dependent on a number of factors. These factors include the desired antimicrobial activity and the like. The antimicrobial composition disclosed herein can be assembled by combining the amphiphilic molecule and the silver nanoparticle using methods well know to those of skill in the art. It is contemplated that the antimicrobial composition disclosed herein can be easily assembled using “click chemistry”, a synthetic philosophy proposed by K.B. Sharpless and coworkers. Reactions of click chemistry are designed to be quick and reliable. Many other attributes of click chemistry include a high chemical yield, the reactions are stereospecific, proceed under simple reaction conditions, have a high atom economy, utilize readily available starting materials and reagents, either no solvent is required involved or a benign solvent can be used, the reagents and products are physiologically stable, and the reactions have a large thermodynamic driving force to favor a reaction with a single reaction product.

It is further contemplated that a variety of click chemistry methods can be utilized to assemble the antimicrobial composition from properly substituting the amphiphilic molecule and the silver nanoparticle with the appropriate functional group. For example, in some embodiments, the antimicrobial composition can be assembled via a dipolarcycloaddition reaction, such as reacting nitrones with alkenes to form isoxazolidines or alkynes with nitriles to form triazoles, other cycloadditions, such as the Diels-Alder, hetero Diels-Alder reaction or [2,2], [2,3], [3,3]-cycloadditions, nucleophilic substitution reactions, such as reacting various nucleophiles with small strained rings like epoxy and aziridine compounds, and carbonyl-chemistry-like formation of esters and ureas.

The most commonly known and utilized click reaction is the 1,3-dipolar cycloaddition reaction of alkynes with nitriles to form triazoles. In one embodiment, the first reactive functional group is an optionally substituted alkyne. In another embodiment, the first reactive functional group is —C≡CH or —CO—C≡CH. In some embodiments, the second reactive functional group is an azide. This embodiment is exemplified in FIG. 1.

In one embodiment, the first reactive functional group is attached to the hydrophobic group. In one embodiment, the second reactive functional group is attached to the silver nanoparticle.

In general, reaction conditions will vary depending on the reagents selected. In some embodiments, the reactions conditions comprise administering heat. The heat can be administered using a number of means, such as a warm water bath, a heat gun or open flame, a hot plate, and the like. In some embodiments, heat is administered by gentle warming with steam towels.

In some embodiments, the assembly of the antimicrobial composition from a substituted amphiphilic molecule and silver nanoparticle can be facilitated by using a catalyst. In some embodiments, the reaction conditions comprise the addition of non-toxic catalyst. The catalyst should not cause adverse health effects or be harmful to mammals, whether the mammal is exposed orally, intravenously or dermally, in the active concentration. In some embodiments, a compound is considered non-toxic based on the LD₅₀ (median lethal dose by oral or dermal exposure) or LC₅₀ (median lethal inhalation concentration for a one-hour exposure) value. It is suggested that non-toxic reagents have an oral LD₅₀ of greater than 500 mg/kg or a dermal LD₅₀ of greater than 1000 mg/kg. In some embodiments, the non-toxic catalyst is tris[(1-benzyl-1H-1,2,3-triazol-4-yl)methyl]amine (TBTA).

### Methods of Using the Silver Nanoparticle-Containing Antimicrobial Composition

Since the discovery of antibiotics, such as penicillin and tetracycline, bacteria have developed a resistance to these as they are able to rapidly mutate. Many strains of resistant bacteria are showing up in hospitals as well as in urban areas. Silver nanoparticles have shown excellent efficacy as an antimicrobial when used to treat wounds and burns. It is postulated that the efficacy stems from the ability of the silver nanoparticles to attack the cell at multiple cites and inactivate critical physiological functions. Using silver nanoparticles as an antimicrobial is particularly attractive because the microbes are not able to mutate to avoid its antimicrobial effect and thus are unable to build resistance. The development of resistance to silver nanoparticles would be extremely rare because an organism would have to undergo simultaneous mutations in every critical function within a single generation to escape the silver's influence.

Disclosed herein is a method of preventing or treating a bacterial infection in a mammalian subject, comprising administering to the subject an effective amount of the antimicrobial composition disclosed herein. In some embodiments, the composition is administered to at least one site of a medical condition on the mammalian subject.

The use of silver to treat medical conditions has been well documented (Capelli U.S. Pat. No. 5,662,913; Nielson et al. U.S. Pat. No. 7,329,417 and Burrell, et al. U.S. Pat. No. 5,958,440, each of which is incorporated herein by reference). In some embodiments, the antimicrobial composition can be used to treat medical conditions such as acne, burn, sunburn, chemical burn, rash, lesion, scrape, blister, scale, abscess, sore, and the like.

In some embodiments, the antimicrobial composition can be used for the manufacture of a medicament to prevent or treat a bacterial infection in a mammalian subject. Also disclosed herein is a kit comprising: a) an antimicrobial composition as disclosed herein; and b) instructions for the use of the antimicrobial composition.

In some embodiments, the antimicrobial composition is formulated for topical administration to skin of a mammalian subject. It is contemplated that various topical formulations can be produced, and methods for the production of such formulations are well known in the art (Hahn, et al. U.S. Pat. No. 7,404,967, which is incorporated herein by reference). The antimicrobial compositions can take the form of a foam, paste, ointment, lotion, cream, gel, salve, solution, suspension, emulsion, powder, pellet, sustained-release formulation, aerosol, spray, or any other form suitable for use. Compositions for topical administration can be either hydrophillic or hydrophobic and can be aqueous or non-aqueous. Compositions for topical administration can be in the form of an emulsion. For topical administration, the compositions typically contain the silver nanoparticle in an amount ranging from about 0.01 to about 60 weight percent of the topical formulation, or alternatively, about 0.01 to about 50 weight percent, or alternatively, about 0.01 to about 40 weight percent, or alternatively, about 0.01 to about 30 weight percent, or alternatively, about 0.01 to about 20 weight percent, or alternatively, about 0.01 to about 15 weight percent, or alternatively, about 0.01 to about 10 weight percent, or alternatively, about 0.01 to about 5 weight percent, or alternatively, about 0.05 to about 20 weight percent, or alternatively, about 0.05 to about 10 weight percent, or alternatively, about 0.05 to about 5 weight percent, of the topical formulation. Additives for topical formulations are well-known in the art, and may be added to the topical composition, as long as they are pharmaceutically acceptable and not deleterious to the epithelial cells or their function. Further, they should not cause deterioration in the stability of the composition. For example, inert fillers, anti-irritants, excipients, percipients, fragrances, pacifiers, antioxidants, gelling agents, stabilizers, surfactants, emollients, coloring agents, preservatives, buffering agents, other permeation enhancers, and other conventional components of transdermal delivery devices as are known in the art.

In one embodiment, the antimicrobial composition is included in a topical cosmetic formulation. In another embodiments, the antimicrobial composition is included in a topical pharmaceutical formulation. In some embodiments, the formulation comprises a pharmaceutically acceptable excipient. Such pharmaceutical excipients can be liquids, such as water and oils, including those of petroleum, animal, vegetable, or synthetic origin, such as peanut oil, soybean oil, mineral oil, sesame oil, and the like. Pharmaceutically acceptable excipients include, but are not limited to, binding agents, filling agents, lubricating agents, suspending agents, sweeteners, flavoring agents, preservatives, buffers, wetting agents, disintegrants, effervescent agents, coloring agents, pH buffering agents, and other excipients depending upon the route of administration and the dosage form desired. Such excipients are known in the art. Examples of suitable pharmaceutical excipients are described in Remington's Pharmaceutical Sciences 1447-1676 (Alfonso R. Gennaro ed., 19th ed. 1995), the contents of which are incorporated herein by reference.

One advantage of using silver nanoparticles to treat infections is the non-toxic nature of the metal. Therefore, it is contemplated that the antimicrobial composition disclosed herein can be used to treat internal infections. In some embodiments, the antimicrobial composition is formulated for transdermal administration to skin of a mammalian subject. Transdermal formulations are well known in the art. For example, a carrier solvent such as dimethylsulfoxide or N,N-dimethylformamide can be used to pull the antimicrobial composition through the dermal layers.

Although the antimicrobial ability of silver nanoparticles is known (Tian, J., et al. Chem Med Chem. (2007) 2(1):129-136, which is incorporated herein by reference), very few methods have been developed for the delivery of such particles to an infected area in order to treat bacterial and other microbial outbreaks. Some known uses of silver nanoparticles include coating a dressing for application to a wound or burn (Paddock, H. N., et al. J. Pediatr. Surg. (2007) 42(1):211-213, which is incorporated herein by reference), and coating a medical device, such as catheter (Roe, D., et al. J. Antimicrob. Chemother. (2008) 61 (4):869-876, which is incorporated herein by reference), to avoid exposure to infection during a medical procedure. However, the treatment of a wound or burn using a silver-coated dressing would impose a great deal of pain on the patient as the dressing must be periodically pulled of and changed. Therefore, it may be desirable to wash or soak a patient in a solution containing the antimicrobial composition disclosed herein. In some embodiments, the antimicrobial composition is formulated for use as a detergent (Brouwn, et al. U.S. Pat. No. 6,894,017 and Boskamp, et al. U.S. Pat. No. 5,583,098, each of which is incorporated herein by reference). The detergent can be formulated for cleaning a mammal or an object. Also disclosed herein are formulations and methods for treating or preventing a microbial outbreak on a surface, such as a bed or chair, and on medical equipment, such as a stethoscope, using the antimicrobial composition disclosed herein. In one embodiment, the detergent can be used to wash the contaminated surface. In order to address one of the main sources of resistant bacterial infections, contamination from hospitals and other healthcare facilities, the antimicrobial composition disclosed herein can be used as a sterilizing agent. In one embodiment, the antimicrobial composition is formulated for use as a sterilizing agent. The sterilizing agent can be in the form of a liquid, gel, semi-solid or solid. In one embodiment, the sterilizing agent is a spray. In another embodiment, the sterilizing agent is a solution. In another embodiment, the sterilizing agent is a powder.

### Formulations

The term “carrier” may be selected from any of the embodiments in this section. The compositions of the present invention can comprise fluid or semi-solid vehicles that may include but are not limited to polymers, thickeners, buffers, neutralizers, chelating agents, preservatives, surfactants or emulsifiers, antioxidants, waxes or oils, emollients, and a solvent or mixed solvent system. The topical compositions useful in the subject invention can be made into a wide variety of product types. These include, but are not limited to, lotions, creams, gels, sticks, sprays, ointments, pastes, foams, mousses, and cleansers. These product types can comprise several types of carrier systems including, but not limited to particles and liposomes. If desired, disintegrating agents can be added, such as the cross linked polyvinyl pyrrolidone, agar or alginic acid or a salt thereof such as sodium alginate. Techniques for formulation and administration can be found in “Remington's Pharmaceutical Sciences.” Mack Publishing Co, Easton, Pa. The formulation can be selected to maximize delivery to a desired target site in the body.

Lotions, which are preparations that are to be applied to the skin surface without friction, are typically liquid or semi-liquid preparations in which finely divided solid, waxy, or liquid are dispersed. Lotions will typically contain suspending agents to produce better dispersions as well as compounds useful for localizing and holding the active agent in contact with the skin, e.g., methylcellulose, sodium carboxymethyl-cellulose, or the like.

Creams containing the active agent for delivery according to the present invention are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington: The Science and Practice of Pharmacy, supra, is generally a nonionic, anionic, cationic or amphoteric surfactant.

Gel formulations can also be used in connection with the present invention. As will be appreciated by those working in the field of topical formulation, gels are semi-solid. Single-phase gels contain macromolecules distributed substantially uniformly throughout the carrier liquid, which is typically aqueous, but also may be a solvent or solvent blend.

Ointments, which are semisolid preparations, are typically based on petrolatum or other petroleum derivatives. As will be appreciated by the ordinarily skilled artisan, the specific ointment base to be used is one that provides for optimum delivery for the active agent chosen for a given formulation, and, preferably, provides for other desired characteristics as well, e.g., emolliency or the like. As with other carriers or vehicles, an ointment base should be inert, stable, nonirritating and non-sensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil emulsions or oil-in-water emulsions, and include, for example, cetyl alcohol, glyceryl monostearate, lanolin and stearic acid.

Useful formulations of the invention also encompass sprays. Sprays generally provide the active agent in an aqueous and/or alcoholic solution which can be misted onto the skin or other surface for delivery. Such sprays include those formulated to provide for concentration of the active agent solution at the site of administration following delivery, e.g., the spray solution can be primarily composed of alcohol or other like volatile liquid in which the drug or active agent can be dissolved. Upon delivery to the skin or other surface, the carrier evaporates, leaving concentrated active agent at the site of administration.

The topical pharmaceutical compositions may also comprise suitable solid or gel phase carriers. Examples of such carriers include but are not limited to calcium carbonate, calcium phosphate, various sugars, starches, cellulose derivatives, gelatin, and polymers such as polyethylene glycols.

The topical pharmaceutical compositions may also comprise a suitable emulsifier which refers to an agent that enhances or facilitates mixing and suspending oil-in-water or water-in-oil. The emulsifying agent used herein may consist of a single emulsifying agent or may be a nonionic, anionic, cationic or amphoteric surfactant or blend of two or more such surfactants; preferred for use herein are nonionic or anionic emulsifiers. Such surface-active agents are described in “McCutcheon's Detergent and Emulsifiers,” North American Edition, 1980 Annual published by the McCutcheon Division, MC Publishing Company, 175 Rock Road, Glen Rock, N.J. 07452, USA, which is incorporated herein by reference.

Examples of high molecular weight alcohols include, but are not limited to, cetearyl alcohol, cetyl alcohol, stearyl alcohol, emulsifying wax, glyceryl monostearate. Other examples are ethylene glycol distearate, sorbitan tristearate, propylene glycol monostearate, sorbitan monooleate, sorbitan monostearate (Span 60), diethylene glycol monolaurate, sorbitan monopalmitate, sucrose dioleate, sucrose stearate (Crodesta F-160), polyoxyethylene lauryl ether (Brij 30), polyoxyethylene (2) stearyl ether (Brij 72), polyoxyethylene (21) stearyl ether (Brij 721), polyoxyethylene monostearate (Myrj 45), polyoxyethylene sorbitan monostearate (Tween 60), polyoxyethylene sorbitan monooleate (Tween 80), polyoxyethylene sorbitan monolaurate (Tween 20) and sodium oleate. Cholesterol and cholesterol derivatives may also be employed in externally used emulsions and promote w/o emulsions.

Examples of nonionic emulsifying agents are those with hydrophile-lipophile balances (HLB) of about 3 to 6 for water in oil system and 8 to 18 for oil in water system as determined by the method described by Paul L. Lindner in “Emulsions and Emulsion”, edited by Kenneth Lissant, published by Dekker, New York, N.Y., 1974, pages 188-190, which is incorporated herein by reference. Examples of other nonionic emulsifiers include but are not limited to “BRIJ 72”, the trade name for a polyoxyethylene (2) stearyl ether having an HLB of 4.9; “BRIJ 721”, the trade name for a polyoxyethylene (21) stearyl ether having an HLB of 15.5, “Brij 30”, the trade name for polyoxyethylene lauryl ether having an HLB of 9.7; “Polawax”, the trade name for emulsifying wax having an HLB of 8.0; “Span 60”, the trade name for sorbitan monostearate having an HLB of 4.7; “Crodesta F-160”, the trade name for sucrose stearate” having an HLB of 14.5.

The topical pharmaceutical compositions may also comprise emollients. Emollients are materials used for the prevention or relief of dryness, as well as for the protection of the skin. Useful emollients include, but are not limited to, cetyl alcohol, isopropyl myristate, stearyl alcohol, and the like. A wide variety of suitable emollients are known and can be used herein. See e.g., Sagarin, Cosmetics, Science and Technology, 2nd Edition, Vol. 1, pp. 32-43 (1972), and U.S. Pat. No. 4,919,934, to Deckner et al., issued Apr. 24, 1990, both of which are incorporated herein by reference in their entirety.

The topical pharmaceutical compositions may also comprise suitable antioxidants, substances known to inhibit oxidation. Antioxidants suitable for use in accordance with the present invention include, but are not limited to, butylated hydroxytoluene, ascorbic acid, sodium ascorbate, calcium ascorbate, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone and tocopherols such as vitamin E, and the like, including pharmaceutically acceptable salts and esters of these compounds.

The topical pharmaceutical compositions may also comprise suitable preservatives. Preservatives are compounds added to a pharmaceutical formulation to act as an anti-microbial agent. Among preservatives known in the art as being effective and acceptable in parenteral formulations are benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof.

The topical pharmaceutical compositions may also comprise suitable chelating agents to form complexes with metal cations which do not cross a lipid bilayer. Examples of suitable chelating agents include ethylene diamine tetraacetic acid (EDTA), ethylene glycol-bis(beta-aminoethyl ether)-N,N,N′,N′-tetraacetic acid (EGTA) and 8-Amino-2-[(2-amino-5-methylphenoxy)methyl]-6-methoxyquinoline-N,N,N′,N′-tetraacetic acid, tetrapotassium salt (QUIN-2).

The topical pharmaceutical compositions may also comprise suitable neutralizing agents used to adjust the pH of the formulation to within a pharmaceutically acceptable range. Examples of neutralizing agents include but are not limited to trolamine, tromethamine, sodium hydroxide, hydrochloric acid, citric acid, and acetic acid.

The topical pharmaceutical compositions may also comprise suitable viscosity increasing agents. These components are diffusable compounds capable of increasing the viscosity of a polymer-containing solution through the interaction of the agent with the polymer. Carbopol Ultrez 10 may be used as a viscosity increasing agent.

When compounds are incorporated into topical formulations the concentration of active ingredient in the formulation may be limited by the solubility of the active ingredient in the chosen solvent and/or carrier. The topical pharmaceutical compositions may also comprise one or more suitable solvents.

Liquid forms, such as lotions suitable for topical administration or suitable for cosmetic application, may include a suitable aqueous or nonaqueous vehicle with buffers, suspending and dispensing agents, thickeners, penetration enhancers, and the like. Solid forms such as creams or pastes or the like may include, for example, any of the following ingredients, water, oil, alcohol or grease as a substrate with surfactant, polymers such as polyethylene glycol, thickeners, solids and the like. Liquid or solid formulations may include enhanced delivery technologies such as liposomes, microsomes, microsponges and the like.

Additionally, the compounds can be delivered using a sustained release system, such as semipermeable matrices of solid hydrophobic polymers containing the therapeutic agent. Various sustained release materials have been established and are well known by those skilled in the art. Sustained-release capsules can, depending on their chemical nature, release the compounds for a few weeks up to over 100 days.

Topical treatment regimens according to the practice of this invention comprise applying the composition directly to the application site, from one to several times daily. In some embodiments, the composition is then washed away with a solution such as water.

Formulations of the present invention can be used to treat, ameliorate or prevent conditions or symptoms associated with bacterial infections, acne, inflammation and the like.

A topical formulation may be prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### EXAMPLES

All numerical designations, e.g., pH, temperature, time, concentration, and molecular weight, including ranges, are approximations which are varied (+) or (−) by increments of 10%. It is to be understood, although not always explicitly stated that all numerical designations are preceded by the term “about”. It also is to be understood, although not always explicitly stated, that the reagents described herein are merely exemplary and that equivalents of such are known in the art.

These and other embodiments of the present technology will readily occur to those of ordinary skill in the art in view of the disclosure herein and are specifically contemplated.

The present technology is further understood by reference to the following examples, which are intended to be purely exemplary of the present technology. The present technology is not limited in scope by the exemplified embodiments, which are intended as illustrations of single aspects of the present technology only. Any methods that are functionally equivalent are within the scope of the present technology. Various modifications of the present technology in addition to those described herein will become apparent to those skilled in the art from the foregoing description and accompanying figures. Such modifications fall within the scope of the appended claims.

### Example 1

### Nanosoap Synthesis

The synthesis of certain embodiments of the antimicrobial composition disclosed herein are shown in schemes 1 to 4, below. Both the amphiphilic molecules and the silver nanoparticles could be readily obtained either synthetically using methods well known to those of skill in the art or from commercial sources (for the amphiphilic molecules, see “Reactions and Synthesis in Surfactant Systems (Surfactant Science)”, John Texter, Ed., Marcel Drekker, Inc., New York, 2001; for the silver nanoparticles, see Solomon, et al. J. Chem. Ed., 2007, 84(2), 322; Sun, et al. Science, 2002, 298, 2176; or American Elements, Los Angeles, Calif., USA).

As shown in schemes 1 to 4, a silver nanoparticle can first be appended with a reactive functional group (e.g. an azide) via contact with a like functionalized-alkanethiol moiety (e.g. 3-azidopropane-1-thiol, which can be prepared by methods known to one of skill in the art, see Voggu, et al. Chem. Phys. Lett., 2007, 443, 118). Contacting the properly functionalized silver nanoparticle with the appropriately functionalized amphiphilic molecule using conventional “click-chemistry” reaction conditions (see Sharpless, et al. Angew. Chem. Int. Ed., 2001, 40, 2004) would provide the antimicrobial composition as described herein.

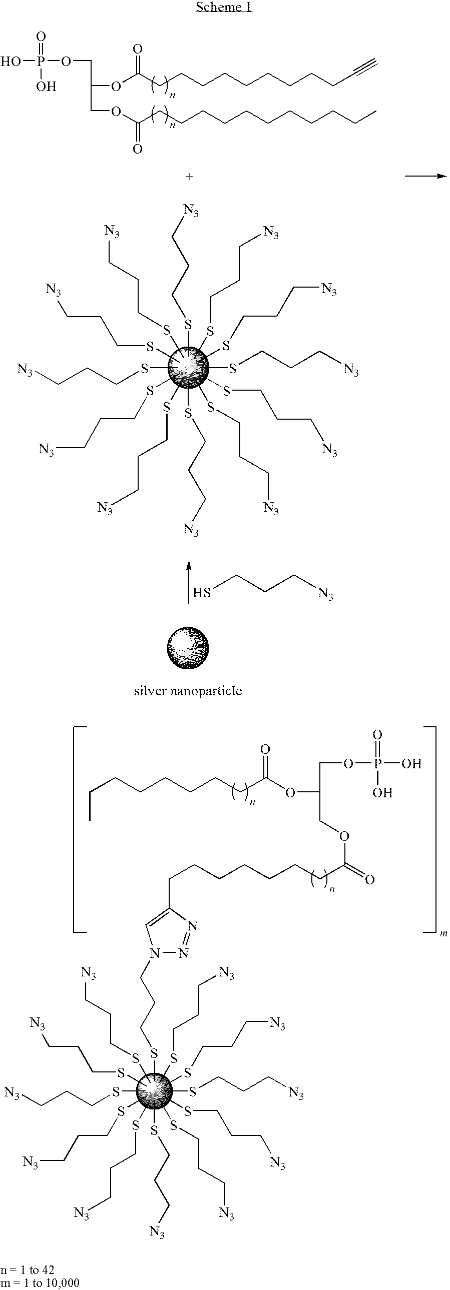

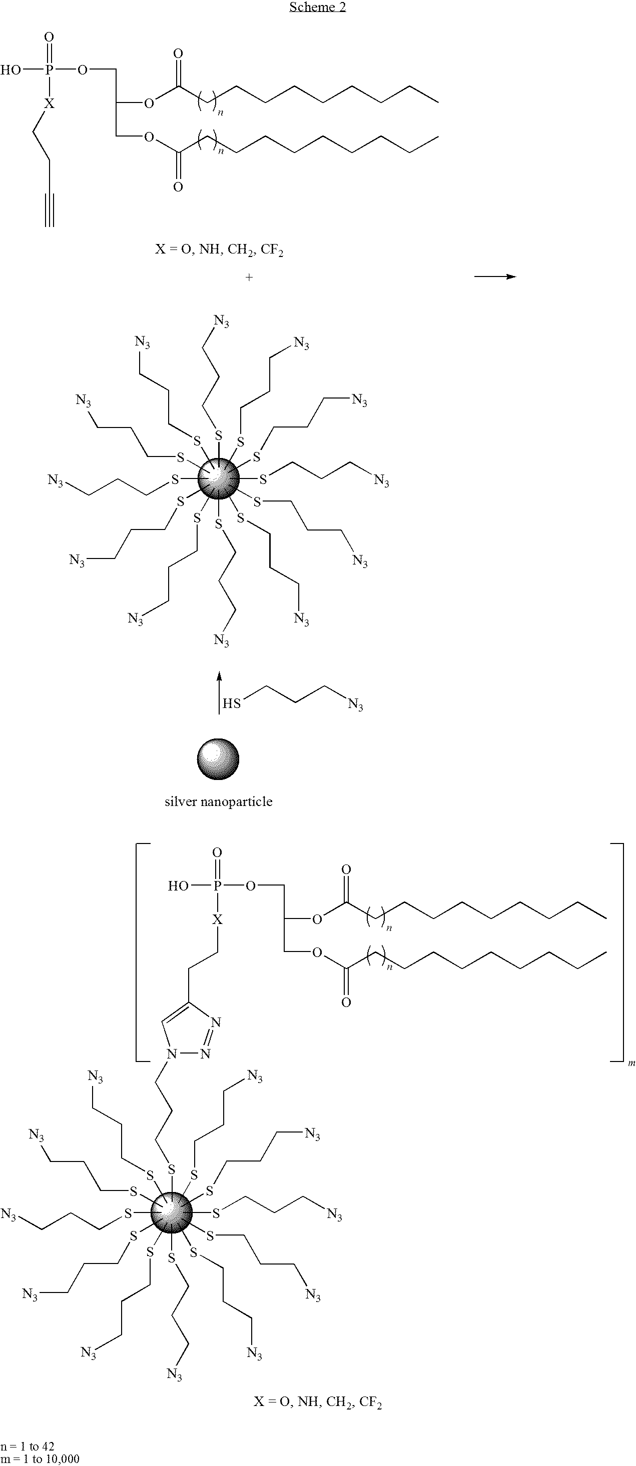

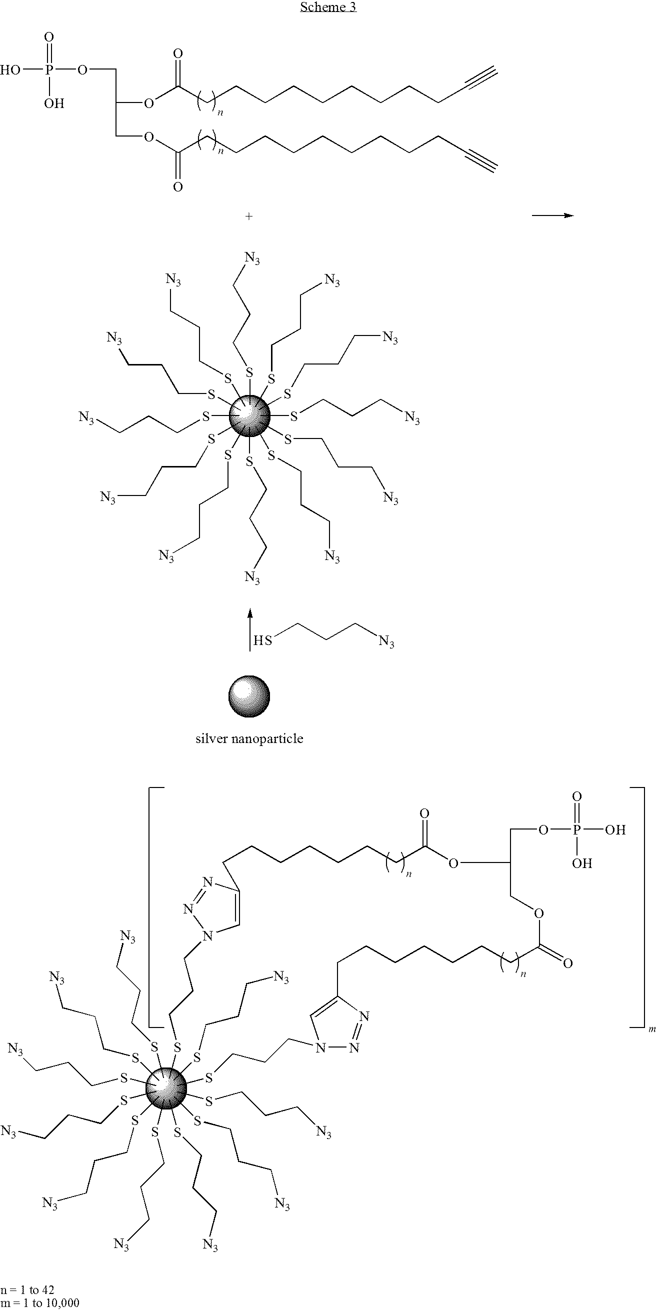

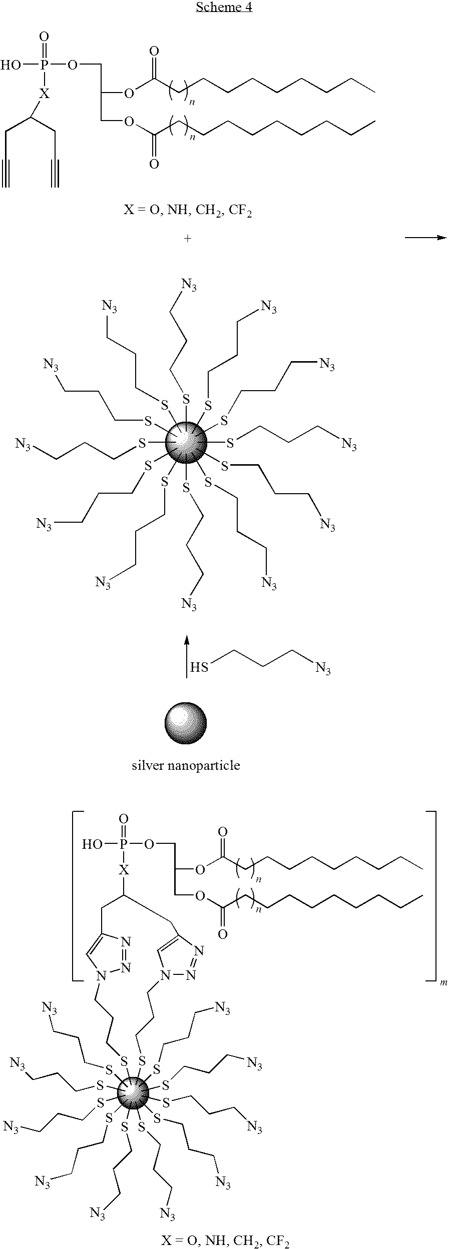

### EQUIVALENTS

The present disclosure is not to be limited in terms of the particular embodiments described in this application, which are intended as single illustrations of individual aspects of. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An antimicrobial composition, comprising:
at least one amphiphilic molecule comprising at least one hydrophilic group and at least one hydrophobic group attached thereto via a linker; and
at least one silver nanoparticle in contact with the amphiphilic molecule.

2. The antimicrobial composition of claim 1, further comprising a carrier.

3. The antimicrobial composition of claim 1, comprising at least two hydrophobic groups.

4. The antimicrobial composition of claim 1, wherein the molecular weight of the antimicrobial composition is from at least about 150 grams per mole to about 50,000 grams per mole.

5. The antimicrobial composition of claim 1, wherein the at least one hydrophobic group are each independently optionally substituted cycloalkyl groups.

6. The antimicrobial composition of claim 5, wherein each cycloalkyl group has from at least about 8 to about 50 carbon atoms.

7. The antimicrobial composition of claim 6, wherein each cycloalkyl group is optionally substituted with from at least about 1 to about 6 substituents selected from the group consisting of alkyl optionally substituted with from 1-3 R⁴ groups, alkenyl optionally substituted with from 1-3 R⁴ groups, alkynyl optionally substituted with 1 R⁴ group, amino, azide, cyano, halo, nitro, silyl, aryl optionally substituted with from 1-4 R⁴ groups, heteroaryl optionally substituted with from 1-3 R⁴ groups, alkoxy, thioalkoxy, carboxy ester, thiocarboxy ester and polyethyleneglycol (PEG), wherein
each R⁴ is independently selected from the group consisting of oxo, thioxo, hydroxyl, amino, azide, cyano, halo, nitro, silyl, alkoxy, thioalkoxy, carboxy ester and thiocarboxy ester.

8. The antimicrobial composition of claim 1, wherein the hydrophilic group is selected from the group consisting of carboxylate; sulfonate; sulfate; sulfinate; phosphate; phosphinate; phosphonate; and quaternary amine.

9. The antimicrobial composition of claim 1, wherein the linker comprises at least one —C—, —NR—, —O—, —S—, —S(O)—, —S(O)₂—, —C(O)—, —C(S)—, —C(NR)—, —C(O)O—, —OC(O)—, —C(O)NR—, —NRC(O)—.

10. The antimicrobial composition of claim 7, wherein the linker comprises at least one —OC(O)—.

11. The antimicrobial composition of claim 1, wherein the silver nanoparticle is bonded to the amphiphilic molecule.

12. The antimicrobial composition of claim 11, wherein the silver nanoparticle is bonded to the amphiphilic molecule through a heterocyclic or heteroaryl linker.

13. The antimicrobial composition of claim 11, wherein the silver nanoparticle is bonded to the hydrophobic group through a heterocyclic or heteroaryl linker.

14. The antimicrobial composition of claim 11, wherein the heterocyclic or heteroaryl linker is selected from the group consisting of triazole, tetrazole, pyrazole, isoxazolidine, pyrrole, isoxazolidinone, oxazole, pyran, furan, benzodiazepine, benzopyran, benzoxazole, indole, benzimidazole, benzotriazole, benzothiadiazole, benzofuroxane, quinoline, quinolidine, quinazoline, benzotriazinone, benzazepine, and fused heterocycles of above mentioned heterocycles.

15. The antimicrobial composition of claim 1, comprising two silver nanoparticles in contact with the amphiphilic molecule.

16. An antimicrobial composition, comprising:
at least one amphiphilic molecule comprising one phosphate group and two alkyl groups attached thereto via a linker; and
one silver nanoparticle bonded to one of the two alkyl groups.

17. The antimicrobial composition of claim 1, formulated for topical administration to skin of a mammalian subject.

18. The antimicrobial composition of claim 1, wherein the antimicrobial composition is included in a topical cosmetic formulation.

19. The antimicrobial composition of claim 1, wherein the antimicrobial composition is included in a topical pharmaceutical formulation.

20. The antimicrobial composition of claim 1, formulated for transdermal administration to skin of a mammalian subject.

21. The antimicrobial composition of claim 1, formulated for use as a detergent.

22. The antimicrobial composition of claim 1, formulated for use as a sterilizing agent.

23. A method for making an antimicrobial composition, comprising contacting at least one amphiphilic molecule comprising at least one hydrophilic group and at least one hydrophobic group attached thereto via a linker having at least one first reactive functional group attached to the at least one hydrophilic group or the at least one hydrophobic group; with at least one silver nanoparticle having a second reactive functional group attached thereto under reaction conditions suitable to form the antimicrobial composition.

24. The method of claim 23, comprising at least two hydrophobic groups.

25. The method of claim 23, comprising at least two silver nanoparticles.

26. The method of claim 23, wherein the first reactive functional group is an optionally substituted alkynyl.

27. The method of claim 23, wherein the first reactive functional group is —C≡CH or —CO—C≡CH.

28. The method of claim 23, wherein the first reactive functional group is attached to the at least one hydrophobic group.

29. The method of claim 23, wherein the second reactive functional group is an azide.

30. The method of claim 23, wherein the reaction conditions comprise administering heat.

31. The method of any one of claim 30, wherein the administering heat comprises gentle warming with steam towels.

32. The method of claim 23, wherein the reaction conditions comprise the addition of non-toxic catalyst.

33. The method of claim 32, wherein the non-toxic catalyst is tris[(1-benzyl-1H-1,2,3-triazol-4-yl)methyl]amine (TBTA).

34. A method of preventing or treating a bacterial infection in a mammalian subject, comprising administering to the subject an effective amount of a composition of claim 1.

35. The method of claim 34, wherein the bacterial infection is on or just below the skin of a mammal and the composition is administered to at least one site of a bacterial infection on the mammalian subject.

36. The method of claim 35, wherein the medical condition is selected from the group consisting of acne, burn, sunburn, chemical burn, rash, lesion, scrape, blister, scale, abscess, sore.

37. The use of a composition of claim 1 for the manufacture of a medicament to prevent or treat a bacterial infection in a mammalian subject.

38. A kit comprising:
a) an antimicrobial composition of claim 1; and
b) instructions for the use of the antimicrobial composition.
